(19) United States
(12) Patent Application Publication
(10) Pub. No.: US 2005/0250961 A1
(43) Pub. Date: Nov. 10, 2005

(54) Process for the preparation of 4-(4-fluorobenzoyl) butyric acid

(76) Inventor: Reddy Muddasani Pulla, Hyderabad (IN)

Correspondence Address: Merchant & Gould, P O Box 2903, Minneapolis, MN, 55402-0903, US

(21) Appl. No.: 10/516,770

(22) PCT Filed: Apr. 16, 2003
(86) PCT No.: PCT/IN03/00159

§ 371 Date: Jun. 24, 2005

(30) Foreign Application Priority Data
Jun. 5, 2002 (IN) 427/MAS/2002

(51) Int. Cl. C07C 51/353

(52) U.S. Cl. 562/459

(57) ABSTRACT

The present invention provides an improved process for the preparation of 4-(4-Fluorobenzoyl)butyric acid of formula (I), which is prepared on a commercial scale using normal quality fluorobenzene (benzene content 300-700 ppm) with the desfluoro analogue impurity at an acceptable level (less than 0.1% by HPLC). The 4-(4-Fluorobenzoyl)butyric acid has the formula (I) is a key raw material for the synthesis of anti-hyperlipoproteinemetic drug ezetimibe.

The present invention relates to an improved process for the preparation of 4-(4-fluorobenzoyl)butyric acid. The 4-(4-fluorobenzoyl)butyric acid has the formula-I given below.

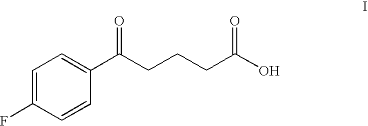

The 4-(4-fluorobenzoyl)butyric acid has the formula-I is a key raw material for the synthesis of anti-hyperlipoproteinemetic drug ezetimibe (U.S. Pat. No. 5,767,115, Schering). The main criticality in making the compound of formula-I lies in controlling the desfluoro analogue impurity (4-benzoylbutyric acid) at an acceptable level (<0.05% W/W by HPLC).

### BACKGROUND OF THE INVENTION

In the literature only two procedures are known for the preparation of compound of formula-I. In the first procedure reported by Compernolle (Tetrahedron, 49, 3193, 1993) fluorobenzene is reacted with glutaric anhydride under Friedel-Crafts conditions using aluminium chloride at 0° C. to get the compound of formula-I in 78% yield. The reaction is done in methylene chloride medium. In this procedure reaction was done on a 15 gr glutaric anhydride scale. The quality of final product with respect to impurities is not addressed in this reference. The main drawbacks in this procedure are:
1. Temperature of the reaction (0° C.) is not suitable for scale up operations. At 0° C. aluminum chloride-glutaric anhydride complex is not soluble in the medium and a thick mass (difficult to stir by mechanical stirrer) will form. Because of improper mixing quality and yield of the product is low.
2. Quenching of Friedel-Crafts reaction by adding water or acid to the reaction mass is difficult on a larger scale.
3. Quality of fluorobenzene used in the process is not mentioned. This is very critical to get an acceptable quality of compound of formula-I with respect to desfluoro analogue impurity.

In the second route (U.S. Pat. No. 6,207,822) for the synthesis of compound of formula-I, fluorobenzene is used as a solvent-cum-reagent in the Friedel-Crafts acylation with glutaric anhydride. The yield of compound of the formula-I reported in this procedure is 79%. The reaction is done at 5-12° C. The main disadvantages in this process are:
1. It requires high quality (benzene content in fluorobenzene should be less than 100 ppm) fluorobenzene to get acceptable quality compound of formula-I.
2. Availability of high quality fluorobenzene is limited for employing the process on a commercial scale.
3. Price of high quality fluorobenzene is almost 4 times higher than the normal quality (benzene content is 300-700 ppm) fluorobenzene. Normal quality fluorobenzene is abundantly available in the market.

Keeping in view of the above mentioned difficulties in implementing the above routes for making compound of formula-I on a commercial scale, we directed our research work to develop a simple, convenient, and economical process for the preparation of compound of formula-I which can also be utilized on a commercial scale

The main objective of the present invention is, therefore, to provide an improved process for the preparation of compound of formula-I as defined above overcoming all the disadvantages present in the hitherto known processes.

Another objective of the present invention is to provide an improved process for the preparation of compound of formula-I as defined above which is simple and economical.

Another objective of the present invention is to provide an improved process for the preparation of compound of formula-I as defined above which does not have any mixing problem when the process is used on any scale of operation.

Another objective of the present invention is to provide an improved process for the preparation of compound of formula-I as defined above which does not have reaction quenching problem when the process is used on any scale of operation.

Still another objective of the present invention is to provide an improved process for the preparation of compound of formula-I as defined above which does not require high quality fluorobenzene.

Another objective of the present invention is to provide an improved process for the preparation of compound of formula-I as defined above which does not require fluorobenzene as solvent medium to carry out the Friedel-Crafts reaction.

### SUMMARY OF INVENTION

In our preliminary studies in the course of the R&D towards development of an improved process for the preparation of compound of formula-I as defined above we found that benzene is more reactive (about 5 times) than fluorobenzene in the above mentioned Friedel-Crafts acylation. This indicates that benzene present in fluorobenzene will react faster than fluorobenzene. Considering this observation we found that
(i) by adjusting the ratio of fluorobenzene with regard to the glutaric anhydride used,
(ii) dividing the amount of fluorobenzene used into two parts—one along with glutaric anhydride and another part with the halogenated solvent and
(iii) fixing the quantity of the halogenated solvent used,
   a process cab be developed for the preparation of compound of the formula-I as defined above which overcomes the drawbacks of the hitherto known processes described above, Further the process developed can also be useful for any commercial scale production of the said compound of the formula-I. The reaction can be conducted at a convenient temperature range of 10 to 25° C.

Accordingly, the present invention provides an improved process for the preparation of compound (4-4(fluorobenzoyl)butyric acid) of the formula-I 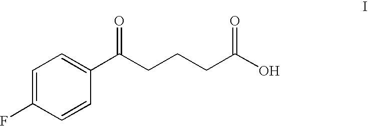
which comprises:
(a) Preparing a solution of normal quality fluorobenzene, glutaric anhydride and halogenated solvent, the amount of fluorobenzene used being in a molar ratio of 0.5 to 0.7 molar equivalent with regard to the amount of glutaric anhydride used.
(b) Preparing a mixture of aluminum chloride, normal quality fluorobenzene and halogenated solvent, the amount of fluorobenzene used being in a molar ratio of 0.5 to 0.6 molar equivalent with regard to the amount of glutaric anhydride used and the amount of halogenated solvent used being at least 4-6 times (w/v) with regard to the amount of glutaric anhydride used.
(c) Adding the solution obtained in step (a) to the mixture obtained in step (b) at a temperature in the range of 10 to 25° C.
(d) Maintaining the resulting reaction mixture at a temperature in the range of 10 to 25° C. for a period in the range of 2 to 4 hrs.
(e) Pouring the reaction mixture into cold dilute hydrochloric acid.
(f) Distilling the halogenated solvent at atmospheric pressure for its recovery.
(g) Filtering and washing the residue with the same halogenated solved used in step (b) above to obtain the compound of the formula-I.
(h) Purifying the compound of the formula-I by dissolving it in aqueous base and precipitating the product by acidification after giving a carbon treatment to the basic solution.
(i) Isolating the precipitated compound of formula-I by filtration and if desired
(j) Recrystallizing the purified acid from a single or mixture of solvents.

The normal quality of fluorobenzene used in step (a) refers to the impurity level of benzene. The benzene content in fluorobenzene may be between 300-700 ppm. The halogenated solvent used in step (b) may be selected form methylene chloride, ethylene dichloride, 1,1,2,2-tetrachloroethane. The base used in step (h) may be selected from sodium carbonate, potassium carbonate, sodium bicarbonate, potassium bicarbonate, sodium hydroxide, potassium hydroxide, and ammonia. The acid used in step (h) may be selected from hydrochloric acid, hydrobromic acid, sulfuric acid, acetic acid, and propionic acid. The solvent used for recrystallization in step (j) may be selected from acetone, methyl ethyl ketone, methyl isobutyl ketone, toluene, acetonitrile, methanol, ethanol, ethyl acetate, hexane or a mixture of these solvents.

The details of the invention are described in Examples given below which are provided to illustrate the invention only and therefore should not be construed to limit the scope of the present invention.

### EXAMPLE 1

### Preparation of 4-(4-fluorobenzoy)lbutyric Acid of Formula-I Using Fluorobenzeile (Benzene Content 300 ppm) with Methylene Chloride as Solvent

Into a 3 L three-necked RB flask were charged 500 ml of methylene chloride, 250 gr of aluminum chloride and 45 gr of fluorobenzene (benzene content 300 ppm) under nitrogen atmosphere. The reaction mixture was cooled to 10° C. and a solution of 100 gr of glutaric anhydride, 45 gr of fluorobenzene (benzene content 300 ppm) and 500 ml of methylene chloride was added slowly over a period of 3 hrs between 10-15° C. The reaction mixture was maintained for another one hour at the same temperature. The reaction mixture was slowly poured onto a mixture of crushed ice (700 gr) and conc. HCI (300 ml) below 10° C. The reaction mass temperature was allowed to reach 25° C. and methylene chloride distilled off from the reaction mixture below 50° C. After cooling the reaction mixture to 20° C., solids were filtered off and washed with 500 ml of water. The wet cake thus obtained was suspended in 250-300 ml of methylene chloride and filtered. The solid compound was dissolved in 600 ml of 4% sodium hydroxide, treated with 10 gr of activated charcoal and filtered. The filtrate was acidified with conc. HCl and the precipitated acid was filtered. After washing the wet cake with 500 ml of water, it was dissolved in 500 ml of acetone. The acetone solution was slowly cooled to 15-20° C. and the solid filtered, washed with chilled acetone (50 ml) and dried at 50-70° C. to get 122 gr of white crystalline solid, m.p. 143° C. Purity by HPLC is 99.65%. Desfluoro impurity is less than 0.05%.

### EXAMPLE 2

### Preparation of 4-(4-fluorobenzoy)lbutyric Acid of Formula-I Using Fluorobenzene (Benzene Content 500 ppm) with Methylene Chloride as Solvent

Into a 3 L three-necked RB flask were charged 500 ml of methylene chloride, 250 gr of aluminum chloride and 45 gr of fluorobenzene (benzene content 500 ppm) under nitrogen atmosphere. The reaction mixture was cooled to 10° C. and a solution of 100 gr of glutaric anhydride, 45 gr of fluorobenzene (benzene content 500 ppm) and 500 ml of methylene chloride was added slowly over a period of 3 hrs between 10-15° C. The reaction mixture was maintained for another one hour at the same temperature. The reaction mixture was slowly poured onto a mixture of crushed ice (700 gr) and conc. HCl (300 ml) below 10° C. The reaction mass temperature was allowed to reach 25° C. and methylene chloride distilled off from the reaction mixture below 50° C. After cooling the reactions mixture to 20° C., solids: were filtered off and washed with 500 ml of water.

The wet cake thus obtained was suspended in 250-300 ml of methylene chloride and filtered. The solid compound was dissolved in 600 ml of 4% sodium hydroxide, treated with 10 gr of activated charcoal and filtered. The filtrate pH was adjusted to 1.0-2.0 with conc. HCl and the precipitated acid of formula-I was filtered. After washing the wet cake with 500 ml of water, it was dissolved in 500 ml of acetone. The acetone solution was slowly cooled to 15-20° C., maintained for 2 h, and the solid filtered, washed with chilled acetone (50ml) and dried at 50-70° C. to get 120 gr of white crystalline solid of formula-I, m.p. 143-143.5° C. Purity by HPLC is 99.7%. Desfluoro impurity is less than 0.05%.

### EXAMPLE 3

### Preparation of 4-(4-fluorobenzoy)lbutyric Acid of Formula-I Using Fluorobenzene (Benzene Content 700 ppm) with Methylene Chloride as Solvent

Into a 3 L three-necked RB flask where charged 500 ml of methylene chloride, 250 gr of aluminum chloride and 45 gr of fluorobenzene (benzene content 700 ppm) under nitrogen atmosphere. The reaction mixture was cooled to 10° C. and a solution of 100 gr of glutaric anhydride, 45 gr of fluorobenzene (benzene content 700 ppm) and 500 ml of methylene chloride was added slowly over a period of 3 hrs between 10-15° C. The reaction mixture was maintained for another one hour at the same temperature. The reaction mixture was slowly poured onto a mixture of crushed ice (700 gr) and conc. HCI (300 ml ) below 10° C. The reaction mass temperature was allowed to reach 25° C. and methylene chloride distilled off from the reaction mixture below 50° C. After cooling the reaction mixture to 20° C., solids were filtered off and washed with 500 ml of water.

The wet cake thus obtained was suspended in 250-300 ml of methylene chloride and filtered. The solid compound was dissolved in 600 ml of 4% sodium hydroxide, treated with 10 gr of activated charcoal and filtered. The filtrate pH was adjusted to 1.0-2.0 with conc. HCl and the precipitated acid of formula-I was filtered. After washing the wet cake with 500 ml of water, it was dissolved in 500 ml of acetone. The acetone solution was slowly cooled to 15-20° C., maintained for 2 h, and the solid filtered, washed with chilled acetone (50 ml) and dried at 50-70° C. to get 123 gr of white crystalline solid of formula-I, m.p. 143° C. Purity by HPLC is 99.6%. Desfluoro impurity is less than 0.05%.

### EXAMPLE 4

### Preparation of 4-(4-fluorobenzoy)lbutyric Acid of Formula-I Using Fluorobenzene (Benzene Content 500 ppm) with Ethylene Dichloride as Solvent

Into a 3 L three-necked RB flask was charged ethylene dichloride (500 ml), aluminum chloride (250 gr) and fluorobenzene (45 gr) under nitrogen atmosphere. The reaction mixture was cooled to 10° C. and a solution of glutaric anhydride (100 gr), fluorobenzene (45 gr) and ethylene dichloride (500 ml) was added slowly over a period of 3 hrs between 10-15° C. After maintaining for one hour at 15-18° C. the reaction mixture was slowly poured onto a mixture of crushed ice (700 gr) and conc. HCl (300 ml) below 10° C. The reaction mass temperature was raised to reach 25° C. and distilled off ethylene dichloride from the reaction mixture below 100° C. After cooling the reaction mixture to 20° C., crude solid was filtered off and washed with 500 ml of water.

The wet cake thus obtained was suspended in 300 ml of ethylene dichloride and filtered. The solid compound was dissolved in 600 ml of 4% sodium hydroxide, treated with 15 gr of activated charcoal and filtered. The filtrate was acidified to pH 1.5-2.0 with conc. HCl and the precipitated acid was filtered. After washing the wet cake with. 500 ml of water, it was dried at 60-70° C. to get 130 gr of white solid, m.p. 140-142° C. This solid was dissolved in 500 ml of acetone. The acetone solution was slowly cooled to 15-20° C. and the solid filtered, washed with chilled acetone (50 ml) and dried at 50-70° C. to get 120 gr of white crystalline solid, m.p. 143° C. Purity by HPLC is 99.65%. Desfluoro impurity is less than 0.05%.

### Advantages of the Present Invention

1. (a) By adjusting the ratio of fluorobenzene with regard to the glutaric anhydride used (b) dividing the amount of fluorobenzene used into two parts—one along with glutaric anhydride and another part with the halogenated solvent and (c) conducting the Friedel-Crafts acylation at 10-20° C., the process can be made applicable to any scale of operation.
2. By using halogenated solvent in the reaction, quality of required fluorobenzene could be relaxed to normal quality to make the process economical.
3. High quality compound of formula-I could be produced using normal quality fluorobenzene.

1. An improved process for the preparation of compound of formula-I 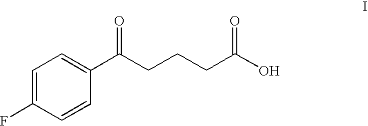
which comprises:
(a) Preparing a solution of normal quality fluorobenzene, glutaric anhydride and halogenated solvent, the amount of fluorobenzene used being in a molar ratio of 0.5 to 0.7 molar equivalent with regard to the amount of glutaric anhydride used.
(b) Preparing a mixture of aluminum chloride, normal quality fluorobenzene and halogenated solvent, the amount of fluorobenzene used being in a molar ratio of 0.5 to 0.6 molar equivalent with regard to the amount of glutaric anhydride used and the amount of halogenated solvent used being at least 4-6 times (w/v) with regard to the amount of glutaric anhydride used.
(c) Adding the solution obtained in step (a) to the mixture obtained in step (b) at a temperature in the range of 10 to 25° C.
(d) Maintaining the reaction mixture at the temperature in the range of 10 to 25° C. for a period in the range of 2 to 4 hrs.
(e) Pouring the reaction mixture into could dilute hydrochloric acid.
(f) Distilling the halogenated solvent at the atmospheric pressure for its recovery.
(g) Filtering and washing the residue with the same halogenated solvent used in step (b) above to obtain the compound of the formula-I.
(h) Purifying the compound of the formula-I by dissolving it in aqueous base and precipitating the product by acidification after giving a carbon treatment to the basic solution.
(i) Isolating the precipitated compound of formula-I by filtration and if desired
(j) Recrystalling the purified acid form a single or mixture of solvents.

2. An improved process for the preparation of compound of formula-I as claimed in claim 1(i) wherein the normal quality fluorobenzene used in the process has a benzene content of 300-700 ppm, preferably between 300-500 ppm.

3. An improved process for the preparation of compound of formula-I as claimed in claim 1 wherein the halogenated solvent used in the reaction is methylene, chloride, ethylene dichloride, 1,1,2,2-tetrachloroethylene, preferably methylene chloride or ethylene dichloride.

4. An improved process for the preparation of compound of formula-I as claimed in claim 1 wherein the quality of solvent used is 6 to 10 times (w/v) on glutaric anhydride, preferably 8 to 10 times.

5. An improved process for the preparation of compound of formula-I as claimed in claim 1 wherein the reaction temperature is between 10-25° C., preferably between 12-18° C.

6. An improved process for the preparation of compound of formula-I as claimed in claim 1 wherein the base used to dissolve the crude acid is ammonia, sodium, carbonate, sodium bicarbonate, sodium hydroxide, potassium carbonate, potassium bicarbonate, potassium hydroxide, ammonia, preferably sodium hydroxide or ammonia.

7. An improved process for the preparation of compound of formula-I as claimed in claim 1 wherein the acid used to neutralize the base is hydrochloric acid.
